(19) United States
(12) Patent Application Publication
(10) Pub. No.: US 2022/0293217 A1
(43) Pub. Date: Sep. 15, 2022

(54) SYSTEM AND METHOD FOR RISK ASSESSMENT OF MULTIPLE SCLEROSIS

(71) Applicant: Tata Consultancy Services Limited, Mumbai (IN)

(72) Inventors: SHARMILA SHEKHAR MANDE, Pune (IN); CHANDRANI BOSE, Pune (IN); HARRISHAM KAUR, Pune (IN)

(73) Assignee: Tata Consultancy Services Limited, Mumbai (IN)

(21) Appl. No.: 17/633,120

(22) PCT Filed: Aug. 4, 2020
(86) PCT No.: PCT/IB2020/057360

§ 371 Date: Feb. 4, 2022

(30) Foreign Application Priority Data
Aug. 5, 2019 (IN) 201921031559

(51) Int. Cl. G16B 30/00; C12Q 1/6869; G16B 40/20; C12Q 1/6874

(52) U.S. Cl. CPC G16B 30/00; C12Q 1/6869; G16B 40/20; C12Q 1/6874

(57) ABSTRACT

Multiple sclerosis (MS) is a neurodegenerative autoimmune disease affecting brain and the spinal cord which results in distorted communication between brain and rest of the body. It is necessary to assess the risk of MS at the earliest. A system and method for diagnosis and risk assessment of an individual for multiple sclerosis has been provided. The system is using a non-invasive method for risk assessment through prediction of metabolic potential of the bacteria residing in gastrointestinal tract of the individual. The system is configured to calculate a score, which is evaluated from the gut bacterial taxonomic abundance profile, indicative of its metabolic potential for production of a particular neuroactive compound. The score is subsequently used to predict the risk of the individual for MS. The present disclosure also provides microbiome based therapeutic approaches that can potentially minimize the side effects through maintaining the healthy cohort of bacteria in gut.

### CROSS-REFERENCE TO RELATED APPLICATIONS AND PRIORITY

The present application claims priority from Indian provisional application no. 201921031559, filed on Aug. 5, 2019. The entire contents of the aforementioned application are incorporated herein by reference.

### TECHNICAL FIELD

The embodiments herein generally relate to the field of multiple sclerosis, and, more particularly, to a method and system for assessing the risk of an individual for multiple sclerosis using the metabolic potential of the resident gut bacteria.

### BACKGROUND

Multiple sclerosis (MS) is a neurodegenerative autoimmune disease affecting brain and the spinal cord. In particular, the immune system attacks the protective myelin sheath surrounding the nerve fibres resulting in distorted communication between brain and rest of the body. In severe cases, the nerve cells themselves may get damaged leading to conditions like paralysis and epilepsy. The common target population of the disease spans 15 to 60 years of age with a higher vulnerability to the young adults. Recently, MS has also been diagnosed in paediatric age group.

The most common type of MS is referred to as Relapsing-Remitting Multiple Sclerosis (RRMS), where the patient experiences periodical occurrence of symptoms. The relapse phase usually develops over days or weeks followed by partial or complete improvement. This in turn is followed by remission phase which may last for months or even years. The diagnostic/screening tests for MS are not very specific and primarily include differential diagnosis which relies on ruling out other disease conditions with similar symptoms. The diagnostic tests include blood test, Magnetic Resonance Imaging (MRI), Lumbar puncture, and evoked potential test. These tests are semi- or highly invasive as well as expensive in nature. All these factors hinder early diagnosis of the disease.

The disease, in present scenario, is incurable. Moreover, the asymptomatic nature of early stages of the disease before the first incidence (of symptoms) makes the treatment challenging. The drugs available at present mostly focus on alleviating the symptoms, speeding up the recovery from attacks, slowing down the disease progression and reducing the rate of relapse.

In addition to that, genetic predisposition to multiple sclerosis is also considered to be a risk factor for development of the disease. Apart from genetic component, many environmental factors like vitamin-D deficiency, viral infection (Epstein Barr virus) have been associated to higher risk of the disease.

The microbial community residing on and within human body is increasingly being acknowledged for its role in health and disease. Disruption in the healthy composition of the microbial community is referred to as dysbiotic condition. A wide range of studies have indicated association between the microbial cohort in the gastrointestinal tract (gut) and various diseases. Alterations in microbial community composition has also been reported in gut samples and brain tissue samples obtained from multiple sclerosis patients compared to those obtained from healthy individuals

### SUMMARY

Embodiments of the present disclosure present technological improvements as solutions to one or more of the above-mentioned technical problems recognized by the inventors in conventional systems. For example, in one embodiment, a system for risk assessment of multiple sclerosis in an individual has been provided. The system comprises a sample collection module, a DNA extractor, a sequencer, one or more hardware processors and a memory. The sample collection module obtains a sample from a body site of the individual. The DNA extractor extracts Deoxyribonucleic Acid (DNA) from the obtained sample. The sequencer sequences the isolated DNA using a sequencer to obtain stretches of DNA sequences. The memory in communication with the one or more hardware processors, wherein the one or more first hardware processors are configured to execute programmed instructions stored in the memory, to: analyze the stretches of DNA sequences to identify a plurality of bacterial taxa present in the sample, wherein the analysis results in the generation of a bacterial abundance profile having a bacterial abundance value of each of the plurality of bacterial taxa in the sample; pre-process the bacterial abundance profile to obtain scaled bacterial abundance values of the bacterial abundance profile; evaluate a score for each bacterial taxa of the plurality of bacterial taxa for producing a set of neuroactive compounds, wherein the set of neuroactive compounds are compounds which influences the functioning of a gut-brain axis and wherein the score is evaluated independently for each compound of the set of neuroactive compounds and stored in a bacteria-function matrix; calculate a metabolic potential (MP) corresponding to each compound of the set of neuroactive compounds using the bacteria function matrix and the scaled bacterial abundance values, wherein the metabolic potential (MP) is indicative of the capability of the bacterial community for producing the neuroactive compound; generate a classification model utilizing the metabolic potential (MP) of each compound of the set of neuroactive compounds using machine learning techniques; predict the risk of the individual to develop or suffering from multiple sclerosis in a significant risk, a low risk or no risk, using the classification model based on a predefined set of conditions; and design therapeutic approaches, through targeting the bacterial groups that are capable of producing a set of neurotoxic compounds or facilitating growth of healthy microbes, wherein the set of neurotoxic compounds are compounds which negatively affects the functioning of the gut-brain axis.

In another aspect, a method for risk assessment of multiple sclerosis in an individual has been provided. Initially, a sample is obtained from a body site of the individual. The Deoxyribonucleic Acid (DNA) is then extracted from the obtained sample. Later, the isolated DNA is sequenced using a sequencer to obtain stretches of bacterial DNA sequences. Further, the stretches of DNA sequences are analyzed to identify a plurality of bacterial taxa present in the sample, wherein the analysis results in the generation of a bacterial abundance profile having a bacterial abundance value of each of the plurality of bacterial taxa in the sample. Further, the bacterial abundance profile is pre-processed to obtain scaled bacterial abundance values of the bacterial abundance profile. Further, a score is evaluated for each bacterial taxa of the plurality of bacterial taxa for producing a set of neuroactive compounds, wherein the set of neuroactive compounds are compounds which influences the functioning of a gut-brain axis and wherein the score is evaluated independently for each compound of the set of neuroactive compounds and stored in a bacteria-function matrix. In the next step, a metabolic potential (MP) corresponding to each compound of the set of neuroactive compounds is calculated using the bacteria function matrix and the scaled bacterial abundance values, wherein the metabolic potential (MP) is indicative of the capability of the bacterial community for producing the neuroactive compound. Further, a classification model is generated utilizing the metabolic potential (MP) of each compound of the set of neuroactive compounds using machine learning techniques. Further, the risk of the individual to develop or suffering from multiple sclerosis in a significant risk, low risk or no risk is predicted using the classification model based on a predefined set of conditions. And finally, therapeutic approaches are designed, through targeting the bacterial groups that are capable of producing a set of neurotoxic compounds or facilitating growth of healthy microbes, wherein the set of neurotoxic compounds are compounds which negatively affects the functioning of the gut-brain axis.

In yet another aspect, one or more non-transitory machine readable information storage mediums comprising one or more instructions which when executed by one or more hardware processors cause risk assessment of multiple sclerosis in an individual. Initially, a sample is obtained from a body site of the individual. The Deoxyribonucleic Acid (DNA) is then extracted from the obtained sample. Later, the isolated DNA is sequenced using a sequencer to obtain stretches of bacterial DNA sequences. Further, the stretches of DNA sequences are analyzed to identify a plurality of bacterial taxa present in the sample, wherein the analysis results in the generation of a bacterial abundance profile having a bacterial abundance value of each of the plurality of bacterial taxa in the sample. Further, the bacterial abundance profile is pre-processed to obtain scaled bacterial abundance values of the bacterial abundance profile. Further, a score is evaluated for each bacterial taxa of the plurality of bacterial taxa for producing a set of neuroactive compounds, wherein the set of neuroactive compounds are compounds which influences the functioning of a gut-brain axis and wherein the score is evaluated independently for each compound of the set of neuroactive compounds and stored in a bacteria-function matrix. In the next step, a metabolic potential (MP) corresponding to each compound of the set of neuroactive compounds is calculated using the bacteria function matrix and the scaled bacterial abundance values, wherein the metabolic potential (MP) is indicative of the capability of the bacterial community for producing the neuroactive compound. Further, a classification model is generated utilizing the metabolic potential (MP) of each compound of the set of neuroactive compounds using machine learning techniques. Further, the risk of the individual to develop or suffering from multiple sclerosis in a significant risk, low risk or no risk is predicted using the classification model based on a predefined set of conditions. And finally, therapeutic approaches are designed, through targeting the bacterial groups that are capable of producing a set of neurotoxic compounds or facilitating growth of healthy microbes, wherein the set of neurotoxic compounds are compounds which negatively affects the functioning of the gut-brain axis.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles:

FIG. 1 illustrates a block diagram of a system for risk assessment of an individual for multiple sclerosis according to an embodiment of the present disclosure.

FIG. 2 depicts the biochemical pathways for production of the six neuroactive compounds in bacteria according to an embodiment of the disclosure.

FIG. 3 is a flowchart illustrating the steps involved in risk assessment of an individual multiple sclerosis according to an embodiment of the present disclosure.

### DETAILED DESCRIPTION

Exemplary embodiments are described with reference to the accompanying drawings. In the figures, the left-most digit(s) of a reference number identifies the figure in which the reference number first appears. Wherever convenient, the same reference numbers are used throughout the drawings to refer to the same or like parts. While examples and features of disclosed principles are described herein, modifications, adaptations, and other implementations are possible without departing from the scope of the disclosed embodiments. It is intended that the following detailed description be considered as exemplary only, with the true scope being indicated by the following claims.

### Glossary—Terms Used in the Embodiments

The expression “microbiome” or “microbial genome” in the context of the present disclosure refers to the collection of genetic material of a community of microorganism that inhabit a particular niche, like the human gastrointestinal tract.

The expression “neuroactive compound” in the context of the present disclosure refers to the compounds that have the capability to regulate/interfere with neurotransmission, thus affecting brain function.

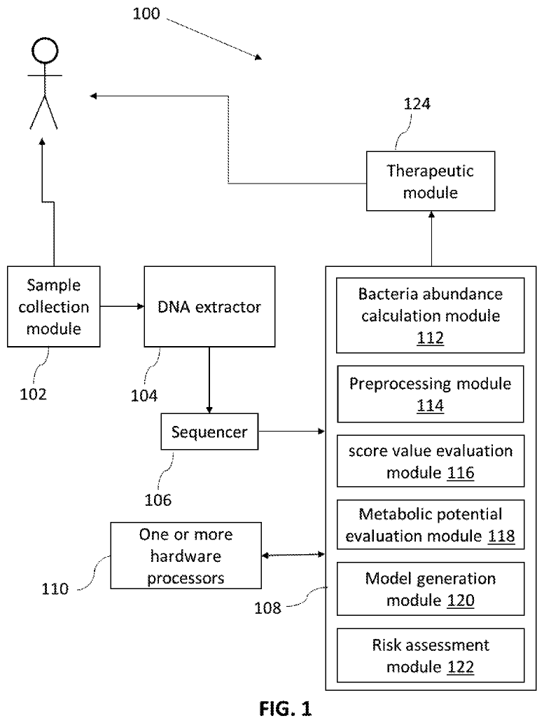

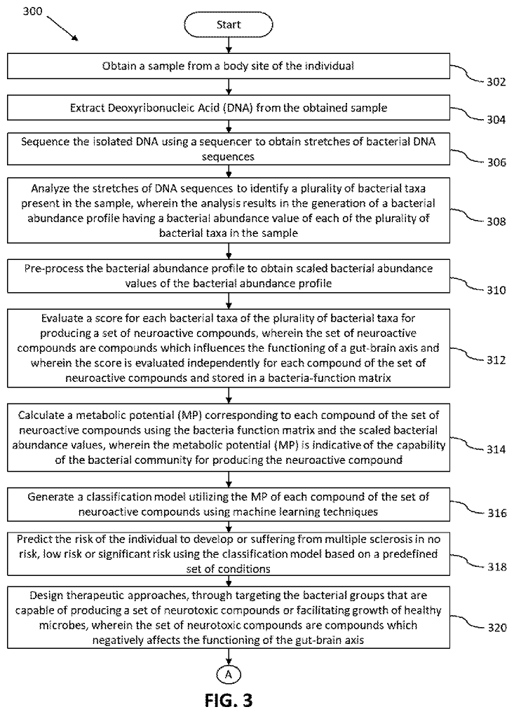

Referring now to the drawings, and more particularly to FIG. 1 and FIG. 3, where similar reference characters denote corresponding features consistently throughout the figures, there are shown preferred embodiments and these embodiments are described in the context of the following exemplary system and/or method.

According to an embodiment of the disclosure, a system 100 for diagnosis and risk assessment of an individual for multiple sclerosis is shown in the block diagram of FIG. 1. The system 100 is using a non-invasive method for risk assessment of the individual for multiple sclerosis through prediction of metabolic potential of the bacteria residing in gastrointestinal tract (gut) of the individual. It should be appreciated that the system 100 is not limited to only bacteria in the gut, other microbes in the gut can also be considered for diagnosis and risk assessment of the individual for multiple sclerosis. Further, the present disclosure also provides microbiome based therapeutic approaches that can potentially minimize the side effects through maintaining the healthy cohort of bacteria in gut.

The system 100 is configured to calculate a score, named as ‘SCORBPEO’ (Score for Bacterial Production of Neuroactive Compounds) is evaluated from the gut bacterial taxonomic abundance profile, which is indicative of its metabolic potential for production of a particular neuroactive compound. It should be appreciated that the score can also be calculated using the abundances of other types of microorganisms. The score is subsequently used to predict the risk of the individual for multiple sclerosis. Given the asymptomatic nature of the disease, the proposed non-invasive approach, if included as a part of routine health screening measures of an individual, can potentially help in early diagnosis of the disease. The system 100 entails targeting the bacterial groups (residing in gut) that are capable of producing neurotoxic compounds or facilitating growth of healthy microbes (including those producing neuro-protective compounds), wherein neuro-protective compounds refer to the compounds which positively affect the functioning of the gut-brain axis.

The present invention relates to systems and methods for non-invasive risk assessment for multiple sclerosis through prediction of metabolic potential of the microbiome residing in gastrointestinal tract (gut). The present invention, in addition, proposes microbiome based therapeutic approaches that can potentially minimize the side effects through maintaining the healthy cohort of bacteria in gut.

According to an embodiment of the disclosure, the system 100 consists of a sample collection module 102, a DNA extractor 104, a sequencer 106, a memory 108 and a processor 110 as shown in FIG. 1. The processor 110 is in communication with the memory 108. The processor 110 is configured to execute a plurality of algorithms stored in the memory 108. The memory 108 further includes a plurality of modules for performing various functions. The memory 108 may include a bacterial abundance calculation module 112, a pre-processing module 114, a score evaluation module 116, a metabolic potential (MP) evaluation module 118, a model generation module 120 and a diagnosis and risk assessment module 122. The system 100 further comprises a therapeutic module 124 as shown in the block diagram of FIG. 1.

According to an embodiment of the disclosure, the microbiome sample is collected using the sample collection module 102. The sample collection module 102 is configured to obtain a sample from a body site of the individual. Normally, the sample is collected in the form of saliva/stool/blood/tissue/other body fluids/swabs from at least one body site/location viz. gut, oral, skin, urinogenital tract etc.

The system 100 further comprises the DNA extractor 104 and the sequencer 106. DNA (Deoxyribonucleic acid) is first extracted from the microbial cells constituting the microbiome sample using laboratory standardized protocols by employing the DNA extractor 104. DNA isolation process using standard protocols based on the isolation kits (like Norgen, Purelink, OMNIgene/Epicentre etc.). Next, sequencing of the microbial DNA is performed using the sequencer 106. The isolated microbial DNA, after purification is subjected to NGS (Next Generation Sequencing) technology for generating human readable form of short stretches of DNA sequence called reads. The said NGS technology involves amplicon sequencing targeting bacterial marker genes (such as 16S rRNA, 23S rRNA, rpoB, cpn60 etc.). The sequence reads, thus obtained, are computationally analysed through widely accepted standard frameworks for NGS data analysis. In another embodiment, the sequencer 106 may involve Whole Genome Sequencing (WGS) where the reads are generated for the total DNA content of a given sample. In yet another embodiment, the set of microbial genes involved in the production of the neuroactive compounds (under the current invention) may be sequenced using targeted PCR (Polymerase Chain Reaction). In yet another implementation, RNA-seq. technology may be used to sequence the microbial RNA (Ribonucleic acid) content of a given sample. This can be performed targeting the whole bacterial RNA content or a particular set of RNAs. RNA-seq provides insights into the active microbial genes in a sample. In the current invention, RNA-seq may be performed targeting the microbial RNAs (or transcripts) corresponding to the set of genes. The extracted and sequenced DNA sequences are then provided to the processor 110.

According to an embodiment of the disclosure, the memory 108 further comprises the bacterial abundance calculation module 112. The bacterial abundance calculation module 112 is configured to the short stretches of DNA sequences to identify a plurality of bacterial taxa present in the sample, wherein the analysis results in the generation of a bacterial abundance profile having a bacterial abundance value of each of the plurality of bacterial taxa in the sample. The generation of bacterial abundance profile involves computationally analyzing one or more of a microscopic imaging data, a flow cytometry data, a colony count and cellular phenotypic data of microbes grown in in-vitro cultures, a signal intensity data, wherein these data are obtained by applying one or more of techniques including culture dependent methods, one or more of enzymatic or fluorescence assays, one or more of assays involving spectroscopic identification and screening of signals from complex microbial populations.

In an example, the bacterial abundance profile is generated, though it should be appreciated that the bacterial abundance module 112 is not limited to only bacteria in the gut, other microbes in the gut can also be considered for analysis. The bacterial abundance calculation module 112 utilizes widely accepted methods/similar frameworks for calculation of abundance profile. The raw abundance profile, thus obtained, is further processed to obtain the relative abundance (RA) of each of the bacterial taxa. The taxa or taxon refers to individual taxonomic groups. Each characterized microbe from the sample can be associated to a taxonomic group. The methodology for calculation of relative abundance (RA) has been provided in the later part of the disclosure.

In the present disclosure, the abundances of the bacterial groups at the taxonomic level of ‘genus’ have been considered. It should be appreciated that in another embodiment, other microbes in the gut can also be considered for diagnosis and risk assessment of the individual for multiple sclerosis. In another embodiment, the abundances of bacterial groups corresponding to other taxonomic levels, such as, but not limited to, phylum, class, order, family, species, strain, OTUs (Operational Taxonomic Units), ASVs (Amplicon Sequence Variant) etc. may be considered.

According to an embodiment of the disclosure, the memory 108 further comprises the pre-processing module 114. The pre-processing module 114 is configured to pre-process the bacterial abundance profile to obtain normalized/scaled bacterial abundance values of the bacterial abundance profile. The pre-processing of the microbial abundance data comprises normalizing to represent the abundance in form of scaled values, wherein the normalization on microbial counts is performed through one or more of a rarefaction, a quantile scaling, a percentile scaling, a cumulative sum scaling or an Aitchison's log-ratio transformation

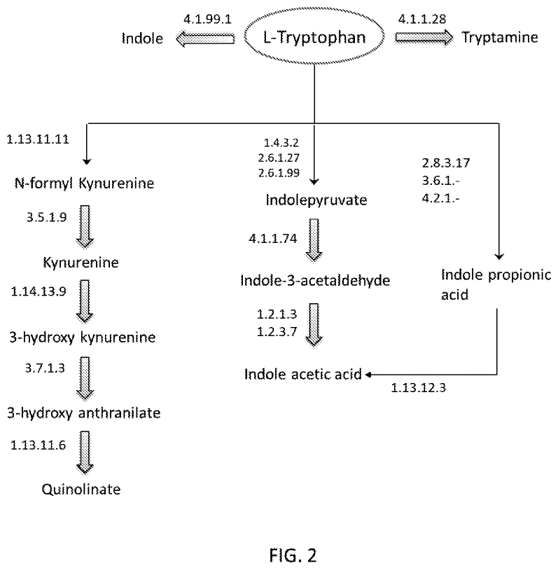

According to an embodiment of the disclosure, the memory 108 further comprises the score evaluation module 116. The score evaluation module 116 is configured to evaluate a score for each bacterial taxa of the plurality of bacterial taxa for producing a set of neuroactive compounds, wherein the set of neuroactive compounds are compounds which influences the functioning of a gut-brain axis and wherein the score is evaluated independently for each compound of the set of neuroactive compounds and stored in a bacteria-function matrix. The gut-brain axis (GBA) refers to a bi-directional link between the central nervous system (CNS) and the enteric nervous system (ENS). The GBA enables communication of emotional and cognitive centres of the brain with peripheral intestinal functions. This communication primarily involves neural, endocrine and immune pathways. It should be appreciated that the function association matrix can also be made using other microorganisms in the gut. In an example, the score can be referred as the “SCORBPEO (Score for Bacterial Production of Neuroactive Compounds)” value. The set of neuroactive compounds include (but not limited to) Kynurenine, Quinolinate, Indole, Indole Acetic Acid (IAA), Indole propionic acid (IPA), and Tryptamine. The biochemical pathways for production of these six compounds (through tryptophan utilization) in bacteria are depicted in FIG. 2.

The ‘SCORBPEO (Score for Bacterial Production of Neuroactive Compounds)’ value for a particular neuroactive compound ‘i’ corresponding to a bacterial genus ‘j’, was calculated using the equation (1)

SCORBPEOᵢⱼ=P*α*β (1)

where P represents the proportion of strains belonging to the genus ‘j’ that have been predicted with compound ‘i’ producing capability (value of P ranges between ‘0’ and ‘1’). Prediction of compound ‘i’ producing capability involves computational identification of the enzymes (proteins) involved in conversion of tryptophan to compound ‘i’. Identification of enzymes was performed using widely accepted tools/packages (such as, but not limited to, Blast, HMMER, Pfam, etc.) which employ protein sequence/functional domain similarity search algorithms. Further, in order to increase the prediction efficiency, a filtration step was included (wherever applicable) based on presence of the genes/functional domains (of a particular pathway) in proximity to each other in the genome of a particular organism.
a denotes a confidence value of the corresponding bacterial group. In an embodiment, the value of a ranges between ‘1’ and ‘10’. It should be appreciated that the value may vary in another embodiments.
β corresponds to a ‘gut weightage’ which represents an enrichment value of a particular pathway in the gut environment. In an embodiment, the value of R ranges between ‘1’ and ‘5’. It should be appreciated that the value may vary in another embodiments. This value is calculated considering the number of gut-strains with capability of producing ‘i’ as compared to the number of corresponding non-gut strains.

In another example, computational identification of enzymes can also be performed using any one or a combination of gene/protein sequence similarity search algorithms, gene′ protein sequence composition based algorithms, protein domain/motif similarity search algorithms, protein structure similarity search algorithms. The enzymes, thus obtained, may further be filtered using any one or a combination of genomic proximity analysis, functional association analysis, catalytic site analysis, sub-cellular localization prediction and secretion signal prediction. Further, identification of enzyme can also be performed using lab experiments which involves enzyme characterization assays.

Thus, in the current example, the values of the computed ‘SCORBPEO’ scores ranged between 0 and 50. The values were further rescaled to ‘0-10’. The range of ‘SCORBPEO’ value and the scaling may vary in another embodiment. For a particular pathway, a bacterial taxon having a higher ‘SCORBPEO’ would indicate a greater probability of production of a particular compound as compared to a taxon with a lower ‘SCORBPEO’.

According to an embodiment of the disclosure, the memory 108 further comprises the metabolic potential (MP) evaluation module 118. The metabolic potential evaluation module 118 is configured to calculate a metabolic potential (MP) corresponding to each compound of the set of neuroactive compounds using the bacteria function matrix and the scaled bacterial abundance values, wherein the metabolic potential (MP) is indicative of the capability of the bacterial community (derived from the sequence data of the extracted DNA) for producing the neuroactive compound. The set of neuroactive compounds include (but not limited to) Kynurenine, Quinolinate, Indole, Indole Acetic Acid (IAA), Indole propionic acid (IPA), and Tryptamine. The metabolic potential (MP) for production of a particular metabolite (by the bacterial community of interest) is calculated based on—(i) the relative abundance of the bacterial genera predicted to have the corresponding metabolic pathway and (ii) a predefined score referred to as (in the current invention) ‘SCORBPEO’ which represents the potential of a particular genus for production of the metabolite. Thus, the MP for production of a particular metabolite by the bacterial community (of interest) can be written as follows in equation (2). The equation (2) has been provided for the calculation of metabolic potential (MP) for Kynurenine.

MP_{Kyn}=Σᵢ₌ⁿRAᵢ×SCORBPEO_{[Kyn][i]} (2)

Where, MP_{Kyn}—Metabolic potential of the bacterial community of interest for the production of Kynurenine. The bacterial community, in the current invention, may indicate the one isolated from the gut sample of the individual.
n—Number of Kynurenine producing bacterial genera present in the bacterial community of interest. This number is acquired from the predefined ‘bacteria-function matrix’. The methodology followed for construction of the ‘bacteria-function matrix’ has been explained in the later part of the disclosure with the help of experimental study.
RA—Relative abundance of a particular bacterial genus predicted to have the metabolic pathway for Kynurenine production. The ‘RA’ is calculated using the pre-processing module 114 as described above.
SCORBPEO_{[Kyn][i]}—The potential of genus ‘i’ for production of Kynurenine as explained earlier

Thus, in the present embodiment, the MP is calculated for six neuroactive compounds, i.e. for Kynurenine, Quinolinate, Indole, Indole Acetic Acid (IAA), Indole propionic acid (IPA), and Tryptamine. These six ‘MP’ values are used further. In an example, the values of the computed MP scores ranges between 0 and 50. Though it should be appreciated that the range of MP values may vary in other examples. The values were further rescaled to ‘0-10’. For a particular pathway, a bacterial taxon having a higher MP would indicate a greater capability of production of a particular compound as compared to a taxon with a lower MP.

It should be appreciated that the MP score or any other score related to bacterial production of any other products/by-products of amino acid metabolism (apart from the above mentioned six compounds) for risk assessment/diagnosis/therapeutics of multiple sclerosis (or any other neurodegenerative disease/disorder) are well within the scope of the present disclosure.

According to an embodiment of the disclosure, the memory 108 further comprises the model generation module 120. The model generation module 120 is configured to a classification model utilizing the metabolic potential (MP) of each compound of the set of neuroactive compounds using machine learning techniques. In an embodiment, the classification model is generated using machine learning techniques using one or more of classification algorithms which include decision trees, random forest, linear regression, logistic regression, naive Bayes, linear discriminant analyses, k-nearest neighbor algorithm, Support Vector Machines and Neural Networks. The model generation module 120 builds the classification model for predicting the risk of the individual to be suffering from multiple sclerosis.

A model for prediction of multiple sclerosis (MS) is generated based on the MP (Metabolic potential) values corresponding to each of the six neuroactive compounds. These six compounds include Kynurenine, Quinolinate, Indole, Indole acetic acid (IAA), Indole propionic acid (IPA), and Tryptamine. The publicly available gut microbiome data (16S rRNA sequences) pertaining to multiple sclerosis patients and matched healthy individuals was used to validate the efficiency of the MS risk assessment scheme proposed in the present disclosure.

According to an embodiment of the disclosure, the memory 108 also comprises the diagnosis and risk assessment module 122. The diagnosis and risk assessment module 122 is configured to predict the risk of the individual to develop or suffering from multiple sclerosis in no risk, a low risk or a significant risk, using the classification model based on a predefined set of conditions. The predefined set of condition comprises comparing the metabolic potential for production of one of the set of neuroactive compounds with a threshold value, wherein the result of comparison is: no risk of multiple sclerosis if the metabolic potential is less than the threshold value, the low risk if the metabolic potential is between the threshold value and a second quartile value of a data set containing the metabolic potential values of the neuroactive compound, and the significant risk if the metabolic potential is more than the second quartile value of a data set containing the metabolic potential values of the neuroactive compound.

For the individuals (of age group 15-60 years) undergoing routine health check-up, especially those with genetic background of the disease, the prediction outcome of the diagnosis and risk assessment module 120 indicates the risk of disease development. For another category of individuals with one or more of the associated symptoms, the diagnosis and risk assessment module 120 can be used as an initial non-invasive diagnostic measure.

According to an embodiment of the disclosure, the system 100 also comprises the therapeutic module 124. The therapeutic module 124 is configured to design therapeutic approaches, through targeting the bacterial groups that are capable of producing a set of neurotoxic compounds or facilitating growth of healthy microbes, wherein the set of neurotoxic compounds are compounds which negatively affects the functioning of the gut-brain axis. The therapeutic module 124 involves identification of a consortium of bacteria/microbes which can be used (in form of pre-/probiotic/synbiotic) in order to—(i) reduce the growth of bacteria (in the gut) which are capable of producing neuroactive (or neurotoxic) compounds and (ii) enhance the growth of beneficial bacteria (in the gut) which can help maintaining a healthy gut or produce neuroactive compounds which are beneficial for functioning and regulation of the gut-brain axis. This consortium of bacteria/microbes can be administered either alone or as an adjunct to the conventional antibiotic drugs for improved therapy of MS, including minimization of the side effects of therapeutic drugs. In the present embodiment, identification of the consortium of bacteria is performed based on the MP values of the bacterial genera identified in a particular sample. Though it should be appreciated that the MP values of other microorganisms can also be considered.

The identification of consortium of bacteria that can potentially facilitate improved therapy of multiple sclerosis (MS) is performed based on the following two aspects—(i) differentially abundant bacterial taxa in cohorts of MS patients and healthy individuals and (ii) the ‘SCORBPEO (Score for Bacterial Production of Neuroactive Compounds)’ values of the differentially abundant taxa corresponding to the production of neuroactive compounds. It should be appreciated that the system 100 is not limited to only bacteria in the gut, other microbes in the gut can also be considered for diagnosis and risk assessment of multiple sclerosis. The differentially abundant taxa (genera in the current invention) in MS and healthy cohorts were identified using state-of-art statistical test (such as but not limited to Welch's t-test). The genera, thus obtained, are listed in the TABLE1 below.

TABLE 1
Differentially abundant bacterial genera in the cohorts
of multiple sclerosis patients and healthy individuals
and their corresponding ‘SCORBPEO’ values for
production of the neuroactive compounds under study

| Bacterial | ‘SCORBPEO’ value | | | |
|---|---|---|---|---|
| Genera | | Indole acetic | Indole propionic | |
| (P-value) | Indole | acid (IAA) | acid (IPA) | Tryptamine |

Differentially abundant bacterial genera having neuroactive
compound producing potential in Healthy cohort

| | | | | |
|---|---|---|---|---|
| Anaerotruncus | 1 | — | — | — |
| Blautia | — | — | — | 0.67 |
| Bacteroides | 3.94 | — | — | — |
| Coprococcus | — | — | — | 0.33 |
| Eggerthella | — | — | 0.2 | — |
| Holdemania | — | — | — | 1 |
| Intestinibacter | — | 1 | — | — |
| Lactobacillus | — | 0.67 | — | — |
| Lactococcus | — | 4.58 | — | — |

Differentially abundant bacterial genera having neuroactive
compound producing potential in Multiple sclerosis cohort

| | | | | |
|---|---|---|---|---|
| Alistipes | 1.5 | — | — | — |

The proposed pre-/probiotic/synbiotic formulation may be composed of IAA and/or IPA producing bacterial genera that are differentially abundant in healthy cohort. In the current example (as shown in TABLE 1) four differential genera (in healthy cohort) namely, Intestinibacter, Eggerthella, Lactobacillus, and Lactococcus have ‘SCORBPEO’ values pertaining to either IAA or IPA. More specifically, the one or more bacterial strains (having ‘SCORBPEO’ values for IAA and/or IPA) belonging to these genera are proposed to be potential probiotic candidates for maintenance of healthy gut microbiome and lowering the probability of development of MS. These bacterial strains are listed in the TABLE2 below. The bacterial strains with known beneficial effects (like butyrate production) are most probable candidates for probiotic formulation. For example, bacterial strains under the groups Eggerthella sp. YY7918, Intestinibacter bartlettii, Lactococcus lactis, and several strains of Lactobacillus have been reported to have beneficial role in the gut. In addition, these bacterial strains may also be provided as probiotic formulation with the conventional drugs in order to maintain a healthier gut microbiome, thus minimizing the side effects of the conventional therapies.

TABLE 2
Bacterial strains (belonging to the four genera Eggerthella,
Intestinibacter, Lactobacillus, and Lactococcus)predicted
with pathways for production of Indole acetic acid (IAA)
or Indole propionic acid (IPA)

| Bacterial Genus | Bacterial Strain |
|---|---|
| Intestinibacter | Intestinibacterbartlettii DSM 16795 |
| Eggerthella | Eggerthella sp. YY7918 strain YY7918 |
| Lactobacillus | Lactobacillus brevis subsp. Gravesensis ATCC 27305 |
| | Lactobacillus buchneri ATCC 11577 |
| | Lactobacillus hilgardii ATCC 8290 |
| | Lactobacillus plantarum subsp. plantarum ATCC |
| | 14917 = JCM 1149 = CGMCC 1.2437 |
| Lactococcus | Lactococcus lactis strain AI06 |
| | Lactococcus lactis subsp. cremoris A76 strain A76 |
| | Lactococcus lactis subsp. cremoris KW2 strain KW2 |
| | Lactococcus lactis subsp. cremoris MG1363 strain |
| | MG1363 |
| | Lactococcus lactis subsp. cremoris NZ9000 strain |
| | NZ9000 |
| | Lactococcus lactis subsp. cremoris SK11 strain SK11 |
| | Lactococcus lactis subsp. cremoris strain 158 |
| | Lactococcus lactis subsp. cremoris strain JM1 |
| | Lactococcus lactis subsp. cremoris strain JM2 |
| | Lactococcus lactis subsp. cremoris strain JM3 |
| | Lactococcus lactis subsp. cremoris strain JM4 |
| | Lactococcus lactis subsp. cremoris strain UC109 |
| | Lactococcus lactis subsp. cremoris UC509.9 strain |
| | UC509.9 |
| | Lactococcus lactis subsp. Lactis |
| | Lactococcus lactis subsp. lactis bv. diacetylactis strain |
| | FM03 |
| | Lactococcus lactis subsp. lactis CV56 strain CV56 |
| | Lactococcus lactis subsp. lactis Il1403 strain IL1403 |
| | Lactococcus lactis subsp. lactis IO-1 strain IO-1 |
| | Lactococcus lactis subsp. lactis KF147 strain KF147 |
| | Lactococcus lactis subsp. lactis KLDS 4.0325 strain |
| | KLDS 4.0325 |
| | Lactococcus lactis subsp. lactis NCDO 2118 strain |
| | NCDO 2118 |
| | Lactococcus lactis subsp. lactis strain 184 |
| | Lactococcus lactis subsp. lactis strain 229 |
| | Lactococcus lactis subsp. lactis strain 275 |
| | Lactococcus lactis subsp. lactis strain A12 |
| | Lactococcus lactis subsp. lactis strain C10 |
| | Lactococcus lactis subsp. lactis strain S0 |
| | Lactococcus lactis subsp. lactis strain UC06 |
| | Lactococcus lactis subsp. lactis strain UC063 |
| | Lactococcus lactis subsp. lactis strain UC08 |
| | Lactococcus lactis subsp. lactis strain UC11 |
| | Lactococcus lactis subsp. lactis strain UC77 |
| | Lactococcus lactis subsp. lactis strain UL8 |

In operation, a flowchart 300 illustrating the steps involved for risk assessment of multiple sclerosis in an individual is shown in FIG. 3. Initially at step 302, the sample is obtained from the body site of the individual. At step 304, Deoxyribonucleic Acid (DNA) is extracted from the obtained sample. Further at step 306, the isolated DNA is sequenced using a sequencer to obtain stretches of bacterial DNA sequences. Further at step 308, the stretches of DNA sequences are analyzed to identify a plurality of bacterial taxa present in the sample, wherein the analysis results in the generation of a bacterial abundance profile having a bacterial abundance value of each of the plurality of bacterial taxa in the sample.

In the next step 310, the bacterial abundance profile is pre-processed to obtain normalized/scaled bacterial abundance values of the bacterial abundance profile. Later at step 312, the score is evaluated for each bacterial taxa of the plurality of bacterial taxa for producing a set of neuroactive compounds, wherein the set of neuroactive compounds are compounds which influences the functioning of a gut-brain axis and wherein the score is evaluated independently for each compound of the set of neuroactive compounds and stored in a bacteria-function matrix.

At next step 314, the metabolic potential (MP) is calculated corresponding to each compound of the set of neuroactive compounds using the bacteria function matrix and the scaled bacterial abundance values, wherein the metabolic potential (MP) is indicative of the capability of the bacterial community for producing the neuroactive compound. At next step 316, the classification model is generated utilizing the metabolic potential (MP) of each compound of the set of neuroactive compounds using machine learning techniques.

At step 318, the risk of the individual to develop or suffering from multiple sclerosis in no risk, a low risk or a significant risk is predicted, using the classification model based on a predefined set of conditions. And finally at step 320, therapeutic approaches are designed, through targeting the bacterial groups that are capable of producing a set of neurotoxic compounds or facilitating growth of healthy microbes, wherein the set of neurotoxic compounds are compounds which negatively affects the functioning of the gut-brain axis.

According to an embodiment of the disclosure, the system 100 for risk assessment of the individual for multiple sclerosis can also be explained with the help of following example.

The prediction of the bacterial community's MP (metabolic potential) score for production of neuroactive compounds requires the bacterial taxonomic abundance data, generated using one of the state-of-art algorithms, as the input. An example of the bacterial taxonomic abundance data has been shown in Table 3. The bacterial taxonomic abundance data has been generated from the gut microbiome data (16S rRNA sequences) provided in the prior art. Gut microbiome data pertaining to a total of 31 multiple sclerosis (MS) patients and 36 matched healthy individuals have been provided in this particular study. A subset of the bacterial abundance data for one MS patient and one healthy individual are shown in the following example in TABLE 3. The abundance of the bacterial taxonomic level genera has been considered in the following example.

TABLE 3
Subset of bacterial genera abundance obtained through
analyzing gut microbiome data corresponding to a multiple
sclerosis patient and a healthy individual.

| | Multiple sclerosis | |
|---|---|---|
| Bacterial Taxa | sample | Healthy sample |
| Alistipes | 248 | 1121 |
| Clostridium_IV | 11 | 228 |
| Coprococcus | 0 | 168 |
| Faecalibacterium | 63 | 885 |
| Flavonifractor | 2 | 23 |
| Fusobacterium | 12 | 0 |
| Prevotella | 38 | 409 |
| Streptococcus | 17 | 0 |

The raw bacterial abundance data is then normalized/scaled to represent the distribution in form of quantile values. Such representation allows easy interpretation of the relative contribution of each taxa in the total bacterial abundance. It should be noted that the use of any kind of normalization or scaling of bacterial abundance values, including percentage, cumulative sum scaling, minmax scaling, maxAbs scaling, robust scaling, percentile, quantile, Atkinson's log transformation, etc. is well within the scope of this disclosure. In the current example, the scaled bacterial abundance includes the decile values of each of the taxa as shown in TABLE4. Scaling to decile values may vary in another embodiment.

TABLE 4
Scaled (decile) values of the bacterial abundances shown in Table 3

| | Multiple sclerosis | |
|---|---|---|
| Bacterial Taxa | sample | Healthy sample |
| Alistipes | 9 | 10 |
| Clostridium_IV | 5 | 8 |
| Coprococcus | 0 | 8 |
| Faecalibacterium | 8 | 10 |
| Flavonifractor | 1 | 6 |
| Fusobacterium | 6 | 0 |
| Prevotella | 8 | 9 |
| Streptococcus | 6 | 0 |

The scaled bacterial genera abundance values are then used to evaluate the score referred to as MP as described above. The present disclosure includes MP scores for six compounds belonging to tryptophan metabolism. These six compounds include, but not limited to, Kynurenine, Quinolinate, Indole, Indole acetic acid (IAA), Indole propionic acid (IPA), and Tryptamine. These compounds have been reported to affect neurological functions through direct or indirect routes.

A model for prediction of multiple sclerosis (MS) is generated based on the ‘MP (Metabolic potential)’ values corresponding to each of the six neuroactive compounds. The publicly available gut microbiome data (16S sequence) pertaining to multiple sclerosis patients and matched healthy individuals is used to validate the efficiency of the MS risk assessment scheme proposed in the current invention. A summary on the datasets used is provided below in TABLE 5:

TABLE 5
Summary of the publicly available microbiome dataset
used for validation of the proposed methodology

| | | No. of MS | No. of healthy |
|---|---|---|---|
| Sample type | Data type | patient | individual |
| Fecal sample | 16S rRNA | 31 | 36 |
| | sequence read | | |

The 16S rRNA data corresponding to the gut microbiome obtained from 31 MS patients and 36 healthy individuals were analyzed in order to obtain the MP values for six above mentioned neuroactive compounds. These values corresponding to a subset of samples is provided in TABLE 6.

TABLE 6
MP values corresponding to the six neuroactive compounds evaluated for a subset
of microbiome samples mentioned in TABLE 5

| | | MP values of six neuroactive compounds | | | | |
|---|---|---|---|---|---|---|
| | | | | | Indole | |
| | | | | | propionic | |
| | | | | | acid (IPA) | |
| | | | | | acid | |
| | Kynurenine | Quinolinate | Indole | Indole acetic | (IAA) | Tryptamine |
| MS_1 | 33 | 10 | 9 | 2 | 1 | 9 |
| MS_2 | 13 | 14 | 10 | 1 | 2 | 5 |
| Healthy_l | 12 | 10 | 10 | 4 | 0 | 7 |
| Healthy_2 | 7 | 4 | 20 | 8 | 0 | 12 |

A model for classification (disease or healthy) of the samples was generated using state-of-art machine learning algorithm considering the six MP values as the feature set for each of the sample. The classification was performed (on the total 67 samples as mentioned in Table 5) for 1000 iterations with randomly chosen 80% of the samples as training set and the remaining 20% as the test set in each iteration. The median MCC (Matthews Correlation Coefficient) value of model training was considered for choosing the best parameters that are able to classify diseased samples from healthy ones. MCC value is a widely accepted measure used in machine learning to indicate the quality of classifications.

In addition to the first feature set consisting of the individual compounds (as mentioned above), another feature set was generated using all possible combination of each feature of the first set, where the value of the combined feature of x and y is equal to (MPₓ+MP_{y}). The second feature set, thus generated was subsequently used to perform a classification. The classification was performed for 1000 iterations with randomly chosen 80% of the samples as training set and the remaining 20% as the test set in each iteration. The features ‘IAA’ and the combination of ‘IAA and IPA’ were observed to classify the samples with higher test sensitivity and specificity. The MCC values, test sensitivities, and test specificities are provided below in TABLE 7.

TABLE 7
Parameters showing model efficiency of classification

| | MCC of | Threshold used | | |
|---|---|---|---|---|
| Feature | training | for testing (T) | Test sensitivity | Test specificity |
| IAA | 82.4 | 1.47 | 92.57 | 88.81 |
| IAA and IPA | 87.88 | 1.49 | 93.72 | 94.39 |

Prediction of risk of multiple sclerosis (MS) could be performed based on the MP values and a threshold value T (corresponding to the two features mentioned in TABLE 7) according to the following rules:

(i) 0<=MP_{IAA}<=T: Significant risk of MS
where, MP_{IAA} is the predicted metabolic potential of the microbiome for IAA production and T is the classification threshold; In the current example T=1.47
   (ii) T<=MP_{IAA}<=Q₂: Low risk of MS
where, Q₂ is the second quartile value of the data set containing MP_{IAA} values; In the current example, Q₂=2 and T=1.47
   (iii) MP_{IAA}>T: No risk of MS
OR
   (i) 0<=MP_{IAA_IPA}<=T: Significant risk of MS where, MP_{IAA_IPA} is the predicted cumulative metabolic potential of the microbiome for IAA and IPA production and T is the classification threshold; In the current example T=1.49
   (ii) T<=MP_{IAA_IPA}<=Q₂: Low risk of MS
where, Q₂ is the second quartile value of the data set containing MP_{IAA_IPA} values; In the current example, Q₂=2 and T=1.49
   (iii) MP_{IAA_IPA}>T: No risk of MS

It may be noted that, the present disclosure primarily relies on the metabolic capability of the resident gut microbiota, which is known to differ in relation to not only the diseased state but also various other factors like dietary pattern, demography, lifestyle etc. Therefore, any other neuroactive compound(s) (or any other compound belonging to amino acid metabolism) either alone or in combination with IAA and IPA may prove to be efficient risk assessment factors for multiple sclerosis or any other neurodegenerative disease for individuals from a different geography or/and of different ethnicity/lifestyle.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments of present disclosure herein address unresolved problem of accurate and early diagnosis of multiple sclerosis. The embodiment provides a system and method for risk assessment of multiple sclerosis in the individual.

It is to be understood that the scope of the protection is extended to such a program and in addition to a computer-readable means having a message therein; such computer-readable storage means contain program-code means for implementation of one or more steps of the method, when the program runs on a server or mobile device or any suitable programmable device. The hardware device can be any kind of device which can be programmed including e.g. any kind of computer like a server or a personal computer, or the like, or any combination thereof. The device may also include means which could be e.g. hardware means like an application-specific integrated circuit (ASIC), a field-programmable gate array (FPGA), or a combination of hardware and software means, e.g. an ASIC and an FPGA, or at least one microprocessor and at least one memory with software processing components located therein. Thus, the means can include both hardware means and software means. The method embodiments described herein could be implemented in hardware and software. The device may also include software means. Alternatively, the embodiments may be implemented on different hardware devices, e.g. using a plurality of CPUs.

The embodiments herein can comprise hardware and software elements. The embodiments that are implemented in software include but are not limited to, firmware, resident software, microcode, etc. The functions performed by various components described herein may be implemented in other components or combinations of other components. For the purposes of this description, a computer-usable or computer readable medium can be any apparatus that can comprise, store, communicate, propagate, or transport the program for use by or in connection with the instruction execution system, apparatus, or device.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope of disclosed embodiments being indicated by the following claims.

1. A method for risk assessment of multiple sclerosis in an individual, the method comprising:
obtaining a sample from a body site of the individual;
extracting Deoxyribonucleic Acid from the obtained sample;
sequencing the isolated DNA using a sequencer to obtain stretches of bacterial DNA sequences;
analyzing, via one or more hardware processors, the stretches of DNA sequences to identify a plurality of bacterial taxa present in the sample, wherein the analysis results in the generation of a bacterial abundance profile having a bacterial abundance value of each of the plurality of bacterial taxa in the sample;
pre-processing, via the one or more hardware processors, the bacterial abundance profile to obtain scaled bacterial abundance values of the bacterial abundance profile;
evaluating, via the one or more hardware processors, a score for each bacterial taxa of the plurality of bacterial taxa for producing a set of neuroactive compounds, wherein the set of neuroactive compounds are compounds which influences the functioning of a gut-brain axis and wherein the score is evaluated independently for each compound of the set of neuroactive compounds and stored in a bacteria-function matrix, wherein the score (SCORBPEO) is calculated using formula:
SCORBPEOᵢⱼ=P*α*β
where P—proportion of strains belonging to the genus ‘j’ that have been predicted with neuroactive compound ‘i’ producing capability,
α—confidence value of the corresponding bacterial group, where the confidence value is evaluated based on the relative number of strains belonging to a particular genus, and
β—‘weightage’ which represents an enrichment value of a particular pathway in a particular body site;
calculating, via the one or more hardware processors, a metabolic potential (MP) corresponding to each compound of the set of neuroactive compounds using the bacteria function matrix and the scaled bacterial abundance values, wherein the metabolic potential (MP) is indicative of the capability of the bacterial community for producing the neuroactive compound, wherein the metabolic potential (MP) is calculated using formula:
$${MP}_{NAC} = {\sum }_{i = 1}^{n} {RA}_{i} \times {SCORBPEO}_{[ NAC ] [ i ]}$$
where, MP_{NAC}—Metabolic potential of the bacterial community (of interest) for production of a particular neuroactive compound,
n—number of the particular neuroactive compound producing bacterial genera present in the bacterial community of interest,
RA—relative scaled abundance of a particular bacterial genus ‘i’ predicted to have the metabolic pathway for the neuroactive compound production, and
SCORBPEO_{[NAC][i]}—The ‘SCORBPEO (Score for Bacterial Production of Neuro-active Compound)’ score of genus ‘i’ for production of the particular neuroactive compound ‘NAC’;
generating, via the one or more hardware processors, a classification model utilizing the metabolic potential (MP) of each compound of the set of neuroactive compounds using machine learning techniques;
predicting, via the one or more hardware processors, the risk of the individual to develop or suffering from multiple sclerosis in a significant risk, low risk or no risk, using the classification model based on a predefined set of conditions; and
designing therapeutic approaches, through targeting the bacterial groups that are capable of producing a set of neurotoxic compounds or facilitating growth of healthy microbes, wherein the set of neurotoxic compounds are compounds which negatively affects the functioning of the gut-brain axis.

2. The method according to claim 1 wherein the predefined set of condition comprises comparing the metabolic potential for production of one of the set of neuroactive compounds with a threshold value, wherein the result of comparison is:
no risk of multiple sclerosis if the metabolic potential is less than the threshold value,
the low risk if the metabolic potential is between the threshold value and a second quartile value of a data set containing the metabolic potential values of the neuroactive compound, and
the significant risk if the metabolic potential is more than the second quartile value of a data set containing the metabolic potential values of the neuroactive compound.

3. The method according to claim 1, wherein the generation of bacterial abundance profile involves computationally analyzing one or more of a microscopic imaging data, a flow cytometry data, a colony count and cellular phenotypic data of microbes grown in in-vitro cultures, a signal intensity data, wherein these data are obtained by applying one or more of techniques including culture dependent methods, one or more of enzymatic or fluorescence assays, one or more of assays involving spectroscopic identification and screening of signals from complex microbial populations.

4. The method according to claim 1, wherein isolating and sequencing stretches of DNA further comprises at least one of:
amplifying and sequencing bacterial 16S rRNA, 23S rRNA, rpoB, or cpn60 marker genes from the bacterial DNA,
amplifying and sequencing one or more of a full-length or one or more specific regions of the bacterial 16S rRNA, 23S rRNA, rpoB, cpn60 marker genes from the microbial DNA,
amplifying and sequencing one or more phylogenetic marker genes from the bacterial DNA, or
whole genome shotgun sequencing (WGS) data corresponding to bacterial DNA, isolated from the body site of the individual.

5. The method according to claim 1, wherein the step of sequencing is performed via one or more of, an amplicon sequencing, a whole genome shotgun sequencing (WGS), a fragment library based sequencing technique, a mate-pair library or a paired-end library based sequencing technique, a polymerase chain reaction (PCR), an RNA sequencing or a microarray-based technique.

6. The method according to claim 1, wherein the step of pre-processing the microbial abundance data comprises normalizing to represent the abundance in form of scaled values, wherein the normalization on microbial counts is performed through one or more of a rarefaction, a quantile scaling, a percentile scaling, a cumulative sum scaling or an Aitchison's log-ratio transformation.

7. The method according to claim 1 wherein the set of neuroactive compounds comprises one or more of Kynurenine, Quinolinate, Indole, Indole acetic acid (IAA), Indole propionic acid (IPA), and Tryptamine.

8. (canceled)

9. (canceled)

10. The method according to claim 1, wherein generating the binary classification model using machine learning techniques may be performed using one or more of random forest, decision trees techniques, linear regression, logistic regression, naive Bayes, linear discriminant analyses, k-nearest neighbor algorithm, Support Vector Machines and Neural Networks techniques.

11. The method according to claim 1, wherein the sample is one of saliva, stool, blood, body fluid, tissue or swab.

12. The method according to claim 1, wherein the body site is one of a gut, oral, skin or urinogenital tract of the individual.

13. The method according to claim 1, wherein the healthy microbes include microbes producing neuro-protective compounds which have beneficial effects on the gut-brain axis.

14. A system for risk assessment of multiple sclerosis in an individual, the method system comprising:
a sample collection module for obtaining a sample from a body site of the individual;
a DNA extractor for extracting Deoxyribonucleic Acid from the obtained sample;
a sequencer for sequencing the isolated DNA using a sequencer to obtain stretches of DNA sequences;
one or more hardware processors; and
a memory in communication with the one or more hardware processors, wherein the one or more first hardware processors are configured to execute programmed instructions stored in the memory, to:
analyze the stretches of DNA sequences to identify a plurality of bacterial taxa present in the sample, wherein the analysis results in the generation of a bacterial abundance profile having a bacterial abundance value of each of the plurality of bacterial taxa in the sample;
pre-process the bacterial abundance profile to obtain scaled bacterial abundance values of the bacterial abundance profile;
evaluate a score for each bacterial taxa of the plurality of bacterial taxa for producing a set of neuroactive compounds, wherein the set of neuroactive compounds are compounds which influences the functioning of a gut-brain axis and wherein the score is evaluated independently for each compound of the set of neuroactive compounds and stored in a bacteria-function matrix, wherein the score (SCORBPEO) is calculated using formula:
SCORBPEOᵢⱼ=P*α*β
where P—proportion of strains belonging to the genus ‘j’ that have been predicted with neuroactive compound ‘i’ producing capability,
α—confidence value of the corresponding bacterial group, where the confidence value is evaluated based on the relative number of strains belonging to a particular genus, and
β—‘weightage’ which represents an enrichment value of a particular pathway in a particular body site;
calculate a metabolic potential (MP) corresponding to each compound of the set of neuroactive compounds using the bacteria function matrix and the scaled bacterial abundance values, wherein the metabolic potential (MP) is indicative of the capability of the bacterial community for producing the neuroactive compound, wherein the metabolic potential (MP) is calculated using formula:
$${MP}_{NAC} = {\sum }_{i = 1}^{n} {RA}_{i} \times {SCORBPEO}_{[ NAC ] [ i ]}$$
where, MP_{NAC}—Metabolic potential of the bacterial community (of interest) for production of a particular neuroactive compound,
n—number of the particular neuroactive compound producing bacterial genera present in the bacterial community of interest,
RA—relative scaled abundance of a particular bacterial genus ‘i’ predicted to have the metabolic pathway for the neuroactive compound production, and
SCORBPEO_{[NAC][i]}—The ‘SCORBPEO (Score for Bacterial Production of Neuro-active Compound)’ score of genus ‘i’ for production of the particular neuroactive compound ‘NAC’;
generate a classification model utilizing the metabolic potential (MP) of each compound of the set of neuroactive compounds using machine learning techniques;
predict the risk of the individual to develop or suffering from multiple sclerosis in a significant risk, a low risk or no risk, using the classification model based on a predefined set of conditions; and
design therapeutic approaches, through targeting the bacterial groups that are capable of producing a set of neurotoxic compounds or facilitating growth of healthy microbes, wherein the set of neurotoxic compounds are compounds which negatively affects the functioning of the gut-brain axis.

15. One or more non-transitory machine readable information storage mediums comprising one or more instructions which when executed by one or more hardware processors cause:
obtaining a sample from a body site of the individual;
extracting Deoxyribonucleic Acid (DNA) from the obtained sample;
sequencing the isolated DNA using a sequencer to obtain stretches of bacterial DNA sequences;
analyzing the stretches of DNA sequences to identify a plurality of bacterial taxa present in the sample, wherein the analysis results in the generation of a bacterial abundance profile having a bacterial abundance value of each of the plurality of bacterial taxa in the sample;
pre-processing the bacterial abundance profile to obtain scaled bacterial abundance values of the bacterial abundance profile;
evaluating a score for each bacterial taxa of the plurality of bacterial taxa for producing a set of neuroactive compounds, wherein the set of neuroactive compounds are compounds which influences the functioning of a gut-brain axis and wherein the score is evaluated independently for each compound of the set of neuroactive compounds and stored in a bacteria-function matrix, wherein the score (SCORBPEO) is calculated using formula:
SCORBPEOᵢⱼ=P*α*β
where P—proportion of strains belonging to the genus ‘j’ that have been predicted with neuroactive compound ‘i’ producing capability,
α—confidence value of the corresponding bacterial group, where the confidence value is evaluated based on the relative number of strains belonging to a particular genus, and
β—‘weightage’ which represents an enrichment value of a particular pathway in a particular body site;
calculating a metabolic potential (MP) corresponding to each compound of the set of neuroactive compounds using the bacteria function matrix and the scaled bacterial abundance values, wherein the metabolic potential (MP) is indicative of the capability of the bacterial community for producing the neuroactive compound, wherein the metabolic potential (MP) is calculated using formula:
$${MP}_{NAC} = {\sum }_{i = 1}^{n} {RA}_{i} \times {SCORBPEO}_{[ NAC ] [ i ]}$$
where, MP_{NAC}—Metabolic potential of the bacterial community (of interest) for production of a particular neuroactive compound,
n—number of the particular neuroactive compound producing bacterial genera present in the bacterial community of interest,
RA—relative scaled abundance of a particular bacterial genus ‘i’ predicted to have the metabolic pathway for the neuroactive compound production, and
SCORBPEO_{[NAC][i]}—The ‘SCORBPEO (Score for Bacterial Production of Neuro-active Compound)’ score of genus ‘i’ for production of the particular neuroactive compound ‘NAC’;
generating a classification model utilizing the metabolic potential (MP) of each compound of the set of neuroactive compounds using machine learning techniques;
predicting the risk of the individual to develop or suffering from multiple sclerosis in a significant risk, low risk or no risk, using the classification model based on a predefined set of conditions; and
designing therapeutic approaches, through targeting the bacterial groups that are capable of producing a set of neurotoxic compounds or facilitating growth of healthy microbes, wherein the set of neurotoxic compounds are compounds which negatively affects the functioning of the gut-brain axis.
